(19) United States
(12) Patent Application Publication
(10) Pub. No.: US 2008/0014162 A1
(43) Pub. Date: Jan. 17, 2008

(54) Method to treat skin in need of a calmative using at least one C-Glycoside derivative

(75) Inventors: Caludie Willemin, Paris (FR); Veronique Burnier, Draveil (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/822,160

(22) Filed: Jul. 2, 2007

Related U.S. Application Data
(60) Provisional application No. 60/836,391, filed on Aug. 9, 2006.

(30) Foreign Application Priority Data
Jul. 3, 2006 (FR) 06 06015

(51) Int. Cl. A61K 31/70; A61K 36/02; A61K 36/48; A61K 36/73; A61K 36/886; A61P 17/00; A61Q 19/00

(52) U.S. Cl. 424/70.1; 424/195.17; 424/725; 424/744; 424/765; 424/773; 424/775; 514/25; 514/53; 514/54

(57) ABSTRACT

The present invention concerns a non-therapeutic method to prevent and/or treat a skin in need of a calmative using a C-glycoside derivative.

This non provisional application claims the benefit of French Application No. 06 06015 filed on Jul. 3, 2006 and U.S. Provisional Application No. 60/836,391 filed on Aug. 9, 2006.

The present invention relates to the non-therapeutic method to prevent and/or treat skin in need of a calmative comprising at least a step of applying to the skin a composition comprising at least one C-glycoside derivative, and also to a dermatological method for preventing and/or treating skin disorders associated with skin irritation comprising at least a step of applying to the skin a composition comprising at least one C-glycoside derivative.

The present invention also relates to a cosmetic or dermatological composition comprising a combination of at least one cosmetic or pharmaceutical compound liable to cause skin irritation and of at least one C-glycoside derivative.

Finally, the present invention relates to a cosmetic process or method comprising at least one step that consists in applying to the skin, a mucous membrane, the scalp and/or the hair a composition according to the invention.

Skin reactions can be manifested in the form of discomfort experienced on the user's skin. They may typically be redness, itching, heating sensations, burning sensations, stinging sensations or tautness. Cosmetic treatments allow this discomfort to be overcome.

Other visible cutaneous signs may also appear, advantageously necessitating in this case a dermatological treatment. Among these visible cutaneous signs, mention may be made especially of pruritus, dry patches, inflammatory erythema, oedema and/or spots, or skin irritation reactions.

These skin reactions, especially skin irritation, may also be induced by exogenous stress, of chemical origin, for example xenobiotics, antigens, allergens, chemical products, compounds liable to cause skin irritation, or peeling treatments, of environmental origin (temperature, climate, UV radiation, atmospheric pollution, especially heavy metals, ozone, cigarette smoke, etc.) or of mechanical origin (friction or shaving) and any stress of endogenous origin such as disorders involving an inflammatory and/or hormonal mechanism affecting the skin, a mucous membrane, the scalp and/or the hair.

Endogenous physiological stress may be linked, for example, to the abnormal production of pro-inflammatory mediators (neuromediators, cytokine or chemokines) or to androgenetic alopecia.

Skin irritation involves a cascade of reactions which, via the recruitment of infiltrating blood cells (neutrophils, macrophages and Langerhans cells) and the substances they release (cytokines, lymphokines, chemokines, etc.), give rise to the persistent irritant process that is mainly characterized by irritation of the skin or an involution of the hair bulb and of its surrounding matrix.

Topical compounds whose use may, under particular circumstances such as a reactive skin, a skin suffering from rosacea, high concentrations of the said compounds, etc., lead to the appearance of skin reactions are used in cosmetic or dermatological compositions, obviously for other effects.

Thus, cosmetic compositions containing, for example, keratolytic and/or desquamating active agents are used for combating ageing, and especially exfoliant active agents and/or active agents that promote cell renewal, such as α-hydroxy acids (especially lactic acid, glycolic acid or citric acid), β-hydroxy acids (especially salicylic acid and 5-n-octanoylsalicylic acid) and retinoids (especially alltrans- or 13-cis-retinoic acid or retinol). Unfortunately, if these active agents are used in excessively large amounts, they may cause skin irritation. The use of these compounds especially by users with irritable and/or allergic skin and/or scalp should thus be limited.

In addition, even certain compounds that are considered as inert in a cosmetic or dermatological composition, for instance preserving agents, surfactants, fragrances, solvents or propellants, may have an irritant nature when they are applied to keratin materials and especially to the skin, including the scalp, of individuals with irritable, sensitive and/or allergic skin, this irritant nature depending on the compound used and on the sensitivity of the skin and the resident cutaneous flora of the user.

Compounds liable to cause skin irritation are generally used in low doses. The use of these compounds in small amount may then prove to be barely advantageous compared with the use of other compounds that are less active, but less irritant or non-irritant and thus used in larger amount, or compared with the intended purpose of the compound, for instance the stability of the composition when it is a matter of emulsifiers or good conservation of the composition when it is a matter of preserving agents.

It is also known that certain skin disorders are associated with a skin reaction of inflammatory or immunoallergic type, among which are inflammatory erythema, psoriasis, cutaneous atopy, atopic dermatitis, allergic reactions of immediate hypersensitivity type, such as urticaria, allergic reactions of delayed hypersensitivity type, such as contact dermatitis or eczema; seborrhoeic dermatitis, acne, inflammatory hyperpigmentations, immune dermatitis, actinic elastosis and pelada or alopecia areata; vitiligo; systemic lupus erythematosus; pemphigus vulgaris; dystrophic bulbous epidermolysis and baldness of autoimmune origin.

There is thus a need to find compounds with a calmative effect capable especially of preventing and/or reducing the irritant effect of cosmetic or dermatological compositions containing one or more compounds capable of causing skin irritation, and of preventing and/or treating skin disorders associated with a skin irritation of inflammatory or immunoallergic type.

The inventors have discovered, surprisingly, that certain C-glycoside derivatives show calmative properties whose advantage especially justifies incorporating them into compositions comprising at least one compound liable to cause skin irritation.

Sugars and sugar derivatives are products that are already exploited for various purposes for the formulation of cosmetic compositions intended either for skincare or for caring for and/or washing keratin fibres.

In particular, certain C-glycoside derivatives are already known for displaying advantageous biological properties, in particular for combating ageing of the epidermis and/or skin dryness. Such compounds are especially described in document U.S. Pat. No. 7,049,300.

Thus, the invention relates to the non-therapeutic method and in particular to a cosmetic method to prevent and/or treat skin in need of a calmative, and especially of a topical calmative, comprising at least a step of applying to the skin a composition comprising at least one C-glycoside derivative.

One subject of the invention is thus the said non-therapeutic method according to the present invention, for preventing and/or treating skin reactions chosen from redness, itching, heating sensations, burning sensations, stinging and tautness.

A subject of the invention is also the said method according to the present invention, wherein the said C-glycoside derivative is intended for preventing and/or reducing the said skin reaction induced by at least one condition chosen from the action of xenobiotics, chemical products, compounds liable to cause skin irritation, a peeling treatment, the action of temperature, the climate, UV radiation or atmospheric pollution, or by friction.

The C-glycoside derivative is then advantageously intended for preventing and/or reducing the irritant effect of a cosmetic or dermatological composition containing one or more compounds liable to cause irritation.

The invention also relates to a non therapeutic, in particular a cosmetic method for the treatment of sensitive skin and/or scalp comprising at least a step of applying to the skin and/or scalp a composition comprising at least one C-glycoside derivative.

The invention moreover relates to a non therapeutic, in particular a cosmetic method for the treatment of sensitive skin and/or scalp comprising at least a step of applying to the skin and/or scalp a composition comprising at least one C-glycoside derivative, wherein it is intended for the treatment of redness and/or darters and/or dysaesthetic sensations chosen among stinging sensations, pins and needles, itching, burning, overheating, discomfort, tautness of of sensitive skin and/or scalp.

According to a particular embodiment, the present invention also relates to a non therapeutic, in particular a cosmetic method for the treatment of sensitive skin and/or scalp comprising at least a step of applying to the skin and/or scalp a composition comprising at least one C-glycoside derivative, wherein the C-glycoside derivative is associated with a rose extract as described hereinafter.

A subject of the invention is also a dermatological method for preventing and/or treating skin disorders associated with skin disorders associated with skin irritation comprising at least a step of applying to the skin a composition comprising at least one C-glycoside derivative wherein skin disorders associated with skin irritation are for example encountered in the case of individuals with irritable and/or allergic skin and/or mucous membranes and/or scalp.

The invention thus relates to C-glycoside derivatives for their use in treating skin disorders associated with skin irritation, for example encountered in the case of individuals with irritable and/or allergic skin and/or mucous membranes and/or scalp.

A subject of the invention is more particularly the said dermatological method according to the invention, wherein the skin irritation is induced by at least one condition chosen from the action of xenobiotics, chemical products, compounds liable to cause skin irritation, a peeling treatment, the action of temperature, the climate, UV radiation or atmospheric pollution, or by friction.

The invention also more particularly relates to the said dermatological method according to the invention, wherein the said C-glycoside derivative is intended for preventing and/or reducing the irritant effect of a cosmetic or dermatological composition containing one or more compounds liable to cause skin irritation.

A subject of the invention is also a dermatological method for preventing and/or treating skin disorders associated with a skin reaction, for example of inflammatory or immunoallergic type, comprising at least a step of applying to the skin a composition comprising at least one C-glycoside derivative.

The invention thus also relates to C-glycoside derivatives for their use in treating skin disorders associated with a skin reaction, for example of inflammatory or immunoallergic type.

Thus, the C-glycoside derivatives are more particularly useful for combating skin disorders chosen from dry patches, oedema and/or spots, inflammatory erythema; pruritus; psoriasis, cutaneous atopy, atopic dermatitis; urticaria; contact dermititis, eczema; seborrhoeic dermatitis; acne; inflammatory hyperpigmentations; immune dermatitis; bullous immune diseases; scleroderma; actinic elastosis; pelada or alopecia areata; vitiligo; systemic lupus erythematosus; pemphigus vulgaris; dystrophic bulbous epidermolysis and baldness of autoimmune origin.

Whether it is for cosmetic or therapeutic purposes, the use according to the present invention is particularly intended for treating sensitive and/or irritable and/or allergic skin and/or mucous membranes and/or scalps.

A subject of the invention is also a composition for topical application to the skin and/or mucous membranes and/or the scalp, comprising, in a physiologically acceptable medium:
at least one cosmetic or pharmaceutical compound liable to cause skin irritation, chosen from desquamating agents, retinoids, urea and derivatives thereof, vitamin D and derivatives thereof, vitamin B9 and derivatives thereof, peroxides, hair-loss counteractants, hair dyes and hair colorants, antiperspirants, deodorants, hair-removing and/or permanent-waving active agents, thioglycolate and salts thereof, phenoxyethanol, 1,2-pentanediol, anthralins, anthranoids, lithium salts, depigmenting agents, certain slimming active agents with a heating effect, nicotinates and derivatives thereof, capsaicin, anti-louse active agents, anti-proliferative agents, antiviral agents, anti-parasitic agents, antifungal agents, anti-pruriginous agents, anti-seborrhoeic agents, certain sunscreens, pro-pigmenting agents, alkyl sulfates and alkyl ether sulfates, cationic or amphoteric surfactants, and
at least one C-glycoside derivative.

In one variant of the invention, the cosmetic or pharmaceutical compound liable to cause skin irritation is neither a keratolytic agent nor a desquamating agent.

A subject of the invention is also a composition for topical application to the skin and/or mucous membranes and/or the scalp, comprising, in a physiologically acceptable medium:
0.00001% to 95% by weight of at least one anti-inflammatory and/or calmative compound, and
at least one C-glycoside derivative.

A subject of the invention is also a cosmetic process for the non-therapeutic care of the skin, the hair and/or the scalp, wherein it comprises at least one step that consists in applying to the skin, the hair and/or the scalp a cosmetic composition according to the present invention.

Finally, a subject of the invention is a cosmetic process for the non-therapeutic care of the skin, the hair and/or the scalp using at least one cosmetic composition according to the present invention, which is particularly suitable for treating sensitive and/or irritable and/or allergic skin and/or mucous membranes and/or scalps.

This cosmetic process according to the invention is especially intended for preventing and/or treating the signs of ageing of the skin, and/or for lightening the skin, and/or for reviving the radiance of the complexion, and/or for stimulating hair growth or preventing hair loss.

According to one variant of the invention, the cosmetic process is intended for soothing the skin, in particular for preventing and/or treating cutaneous manifestations chosen from redness, itching, heating sensations, burning sensations, stinging and tautness.

More particularly, the process according to the invention is intended for soothing the skin of individuals to whom a product liable to cause skin irritation has been administered and especially applied topically.

Unless otherwise indicated, the term “treating” means any action directed towards improving the comfort or well-being of an individual, this term thus covering not only preventing, attenuating and relieving but also curing.

The use of at least one C-glycoside derivative thus has, inter alia, the advantage of eliminating the skin irritation that the compounds with an irritant side effect might have caused, and also of allowing an increase in the amount of the said compounds in cosmetic or dermatological compositions compared with the amount normally used, for the purpose of increased efficacy of these compounds.

Thus, it is possible to use, including in compositions intended for sensitive and/or irritable and/or allergic skin and/or mucous membranes and/or scalps, agents liable to cause skin irritation such as cosmetic active agents (e.g.: keratolytic and/or desquamating agents), dermatological active agents (e.g.: retinoids), certain surfactants, preserving agents, fragrances, solvents and propellants, and mixtures thereof provided that the said compositions comprise at least one C-glycoside derivative.

For the purposes of the invention, the term “skin” means any area of bodily skin including the skin and broadened to the scalp and mucous and semi-mucous membranes.

It has been demonstrated that substance P may be responsible for skin manifestations that characterize sensitive skin.

In general, sensitive skin is defined by a specific reactivity of the skin.

This skin reactivity is conventionally reflected by the manifestation of signs of discomfort in response to the individual coming into contact with a triggering element which may have various origins. It may be the application of a cosmetic product to the surface of the sensitive skin, the ingestion of food products, exposure to abrupt temperature variations, to atmospheric pollution and/or to ultraviolet or infrared rays. Related factors such as age and skin type also exist. Thus, sensitive skin is more common among dry or greasy skin than among normal skin.

The appearance of these signs of discomfort, which appear within minutes following the individual coming into contact with the triggering element, is one of the essential characteristics of sensitive skin. They involve mainly dysaesthesic sensations. The term “dysaesthesic sensations” is intended to mean more or less painful sensations felt in an area of skin, such as stinging, pins and needles, itching, burning, overheating, discomfort, tautness, etc. These subjective signs most commonly exist in the absence of visible clinical signs such as redness and desquamations. It is today known that these skin intolerance and irritation reactions are in particular related to a release of neuropeptides by the epidermal and dermal nerve endings.

In contrast with skin described as allergic, the reactivity of sensitive skin is not the result of an immunological process. Its response mechanism is termed “aspecific”. It is, in this respect, to be distinguished from skin that shows inflammatory and allergic reactions of dermatosis, eczema and/or ichtyosis type, and with respect to which a certain number of treatments have already been proposed.

For obvious reasons, the absence of visible signs makes it difficult to diagnose sensitive skin. Most commonly, this diagnoses is based on questioning the patient. This symptomatology also has the advantage of making it possible to differentiate sensitive skin from contact irritation or allergy for which, on the other hand, visible inflammatory signs exist.

Consequently, the development of “sensitive skin” products has required the provision of tools for evaluating the sensorial reaction of the skin. The inspiration for the first tools, starting with their design, came from the essential characteristic of sensitive skin, namely the presence of signs of discomfort induced by a topical application.

Thus, the lactic acid “stinging test” was the first test proposed. It is carried out by recording stinging sensations reported by a volunteer after application of a 10% lactic acid solution to the sides of the nose. Individuals reporting moderate or strong stinging sensations are called “stingers” and are considered to have sensitive skin. Because of this sensitivity of the skin to the topical application of a product, these individuals are than selected for testing “sensitive skin” products.

More recently, in order to specifically activate the peripheral nerve endings involved in discomfort, and called nociceptors, recently identified as being involved in sensitive skin, new tests have been proposed that in fact use other inducers of discomfort such as capsaicin.

This second type of test, described in application EP 1 374 913, also constitutes another particularly useful means for the diagnosis of sensitive skin.

For the purpose of the present invention, sensitive skin can cover irritable and/or allergic skin and intolerant skin.

Intolerant skin is skin that reacts to various factors, such as the application of cosmetic or dermatological products or soap, through sensations of overheating, tautness, pins and needles and/or redness. In general, these signs are associated with erythema and with hyperseborrheic or acneic skin, or even skin exhibiting rosacea, with or without sores.

Irritable skin is skin that reacts through pruritus, i.e. through itching or stinging, to various factors such as the environment, emotions, foods, the wind, rubbing, shaving, hard water with a high calcium concentration, temperature variations, humidity or wool.

“Sensitive” scalps have a more unambiguous clinical semiology: the itching and/or stinging and/or overheating sensations are essentially triggered by local factors such as rubbing, soap, surfactants, hard water with a high calcium concentration, shampoos or lotions. These sensations are also sometimes triggered by factors such as the environment, emotions and/or foods. An erythema and hyperseborrhae of the scalp and also dandruff are frequently associated with the above signs.

The applicant has demonstrated the substance P antagonist character of certain C-glycoside derivatives. These compounds are therefore interesting for their use in cosmetic as agent for treating the signs of a sensitive skin.

The composition according to the invention comprises a physiologically acceptable medium, i.e. a medium that is compatible with any keratin material such as the skin, the scalp, the nails, mucous membranes, the eyes and the hair or any other area of bodily skin. This composition may be a cosmetic or dermatological composition and may thus comprise a cosmetically or pharmaceutically acceptable medium.

Relative to the claimed subjects, whether it is a matter of the non-therapeutic use or of the use for therapeutic purposes of the cosmetic process or of the compositions, the preferred mode of administration is the topical route, i.e. application to the skin.

Cosmetic or Pharmaceutical Ingredients Liable to Cause Skin Irritation

The non-therapeutic use according to the present invention is especially directed towards preventing and/or reducing the irritant effect of a cosmetic or dermatological composition containing one or more compounds liable to cause skin irritation.

A composition according to the present invention comprises at least one cosmetic or pharmaceutical compound liable to cause skin irritation.

Among these compounds, mention may be made especially of cosmetic compounds or active agents, dermatological compounds or active agents, surfactants, especially anionic surfactants, preserving agents, detergents, fragrances and especially fragrancing alcoholic solutions, solvents and propellants, and mixtures thereof.

Dermatological or cosmetic active agents that may be mentioned more particularly include certain desquamating agents, which may also be peeling agents.

Among the agents specifically for peeling, mention may be made of abrasive/exfoliant particles of mineral, organic, natural or synthetic origin. Mention may be made more particularly of pumice particles, silica, polyethylene or nylon beads and fruit kernel powders.

Among these desquamating agents, the following are liable to cause skin irritation: saturated monocarboxylic acids (acetic acid) and unsaturated monocarboxylic acids, saturated and unsaturated dicarboxylic acids, saturated and unsaturated tricarboxylic acids; α-hydroxy acids and β-hydroxy acids of monocarboxylic acids; α-hydroxy acids and β-hydroxy acids of dicarboxylic acids; α-hydroxy acids and β-hydroxy acids of tricarboxylic acids, keto acids, α-keto acids or β-keto acids of polycarboxylic acids, of polyhydroxy monocarboxylic acids, of polyhydroxy dicarboxylic acids and of polyhydroxy tricarboxylic acids.

Among the α-hydroxy acids or esters thereof, mention may be made particularly of: glycolic acid, dioic acids, for instance octadecenedioic acid or Arlatone dioc DCA sold by the company Uniqema, citric acid, lactic acid, tartaric acid, malic acid or mandelic acid, and esters thereof, for instance dialkyl (C12/C13) tartrates or Cosmacol ETI, and branched C12-13 trialcohol citrates or Cosmacol ECI sold by the company Sasol.

Among the β-hydroxy acids, mention may be made of: salicylic acid and derivatives thereof (including 5-n-octanoylsalicylic acid).

Among the α-keto acids, mention may be made of ascorbic acid and derivatives thereof.

Among the other desquamating agents, mention may be made of: pyruvic acid, gluconic acid, glucuronic acid, oxalic acid, malonic acid, succinic acid, acetic acid, gentisic acid, cinnamic acid, azelaic acid, phenol; resorcinol; urea and derivatives thereof, hydroxyethylurea or Hydrovance® from National Starch; oligofucoses; jasmonic acid and derivatives thereof; ascorbic acid and derivatives thereof, trichloroacetic acid; extract of Saphora japonica and resveratrol.

Among the desquamating agents, those capable of acting on the enzymes involved in desquamation or comeodesmosome degradation may also be liable to cause skin irritation.

Among these, mention may be made especially of mineral salt chelating agents such as EDTA; N-acyl-N,N′,N′-ethylenediaminetriacetic acid; aminosulfonic compounds and in particular (N-2-hydroxyethylpiperazine-N-2-ethane)sulfonic acid (HEPES); 2-oxothiazolidine-4-carboxylic acid (procysteine) derivatives; derivatives of α-amino acids of glycine type (as described in EP 0 852 949, and also sodium methyl glycine diacetate sold by BASF under the trade name Trilon M®); honey; sugar derivatives such as O-octanoyl-6-D-maltose, O-linoleyl-6-D-glucose and N-acetylglucosamine.

Retinoids are also compounds liable to cause skin irritation. Examples thereof that may be mentioned include retinol and esters thereof, retinal, retinoic acid and derivatives thereof such as those described in documents FR-A-2 570 377, EP-A-199 636, EP-A-325 540 and EP-A-402 072, and adapalene.

Salts and derivatives, for instance cis or trans forms, racemic mixtures, and dextrorotatory or laevorotatory forms of the compounds mentioned above are also considered as compounds liable to cause skin irritation.

Other dermatological or cosmetic active agents liable to cause skin irritation are also mentioned below:
urea and derivatives thereof, for instance hydroxyethylurea or Hydrovance® from National Starch,
certain vitamins such as vitamin D and derivatives thereof such as vitamin D3 and vitamin D2, calcitriol, calcipotriol, tacalcitol, 24,25-diOH vitamin D3,1-OH vitamin D2 and 1,24-diOH vitamin D2; vitamin B9 and derivatives thereof,
peroxides, for instance benzoyl peroxide and hydrogen peroxide,
hair-loss counteractants such as minoxidil and derivatives thereof such as aminexyl,
hair dyes and hair colorants, for instance aminophenols and derivatives thereof such as para-phenylenediamine (p-PDA), N-phenyl p-PDA, 2,5-toluenediamine sulfate, meta-phenylenediamine (m-PDA), 3,4-toluenediamine and ortho-phenylenediamine (o-PDA),
antiperspirants, for instance aluminium salts such as aluminium hydroxychloride,
deodorants,
hair-removing and/or permanent hair-waving active agents such as thioglycolates or aqueous ammonia,
thioglycolate and salts thereof,
phenoxyethanol,
1,2-pentanediol,
fragrancing alcoholic solutions (fragrances, eaux de toilette, aftershaves or deodorants),
anthralins (dioxyanthranol),
anthranoids (for example those described in document EP-A-3 19 028),
lithium salts,
depigmenting agents (e.g.: hydroquinone, vitamin C at high concentration, or kojic acid),
certain slimming active agents with a heating effect,
nicotinates and derivatives thereof,
capsaicin,
anti-louse active agents (pyrethrin),
anti-proliferative agents such as 5-fluorouracil or methotrexate,
antiviral agents,
anti-parasitic agents,
antifungal agents,
anti-pruriginous agents,
anti-seborrhoeic agents,
certain sunscreens,
pro-pigmenting agents such as psoralenes and methylangecilines, and
mixtures thereof.

Preserving agents that may be mentioned include phenoxyethanol, chlorhexidine and benzalkonium chloride.

Surfactants that may be mentioned include anionic, cationic and amphoteric surfactants, more particularly anionic surfactants such as alkyl sulfates and alkyl ether sulfates, for instance lauryl sulfate and lauryl ether sulfate, and salts thereof, especially the sodium salt.

According to one preferred embodiment of the invention, the compound liable to cause skin irritation is chosen from retinoids, α-hydroxy acids, β-hydroxy acids, saturated and unsaturated dicarboxylic acids such as octadecenedioic acid or Arlatone DIOC DCA sold by the company Uniqema, anionic, cationic or amphoteric surfactants, 5-n-octanoylsalicylic acid, antiperspirant active agents such as aluminium salts, (N-2-hydroxyethylpiperazine-N-2-ethane)sulfonic acid (HEPES) and cinnamic acid, and mixtures thereof.

The compound liable to cause skin irritation may be present in the composition according to the present invention in an amount that is sufficient to cause a skin irritation reaction. By way of example, it may be present in a content ranging from 0.0001% to 70% by weight, preferably from 0.01% to 50% by weight and better still from 0.1% to 30% by weight relative to the total weight of the composition.

C-Glycoside

A C-glycoside derivative that is suitable for use in the invention may be a compound of general formula (I) below: 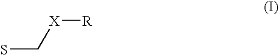
in which:

R represents:
a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
a saturated C₁-C₂₀ and in particular C₁-C₁₀ or unsaturated C₂-C₂₀ and in particular C₂-C₁₀, or saturated or unsaturated, branched or cyclic C₃-C₂₀ and in particular C₃-C₁₀ linear hydrofluoroalkyl or perfluoroalkyl radical; the hydrocarbon-based chain constituting the said radicals possibly being, where appropriate, interrupted with 1, 2, 3 or more heteroatoms chosen from:
   an oxygen,
   a sulfur,
   a nitrogen, and
   a silicon,
      and possibly being optionally substituted with at least one radical chosen from:
   —OR₄,
   —SR₄,
   —NR₄R₅,
   —COOR₄,
   —CONHR₄,
   —CN,
   a halogen atom,
   a C₁-C₆ hydrofluoroalkyl or perfluoroalkyl radical, and/or
   a C₃-C₈ cycloalkyl radical,
      with R₄ and R₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ linear alkyl, perfluoroalkyl or hydrofluoroalkyl radical; or a C₆-C₁₀ aryl radical,
X represents a radical chosen from the groups: 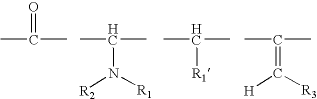
   with R₁, R₂ and R₃ representing, independently of each other, a hydrogen atom or a radical R, with R as defined above, and R_{1′} represents a hydrogen atom, an —OH group or a radical R as defined above, R₁ possibly also denoting a C₆-C₁₀ aryl radical;
—S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be α or β, and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, the said monosaccharide or polysaccharide containing at least one hydroxyl function that is mandatorily free and/or optionally one or more amine functions that are mandatorily protected, X and R otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-p-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (I) for which X represents a group chosen from —CO—, —CH(OH)—, —CH(NR₁R₂)— and —CH(R)—, in particular —CO—, —CH(OH)—, —C(NH₂)—, —C(NHCH₂CH₂CH₂OH)—, —C(NHPh)- and —C(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (I) for which R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X otherwise conserving all the definitions given above. Preferably, R denotes a linear C₁-C₄ and especially C₁-C₃ radical, optionally substituted with —OH, —COOH or —COOR″₂, R″₂ being a saturated C₁-C₄ alkyl radical, especially ethyl.

Preferentially, R denotes an unsubstituted linear C₁-C₄ and especially C₁-C₂ alkyl radical, in particular ethyl.

Among the C-glycoside derivatives of formula (I) that are preferably used are those for which:
R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X represents —CO—, —CH(OH)—, —CH(NR₁R₂)— or —CH(R)—, as defined above.

Preferably, a C-glycoside derivative of formula (I) is used, for which:
R denotes a linear C₁-C₄ and especially C₁-C₃ radical, optionally substituted with —OH, —COOH or —COOR″₂, R″₂ being a saturated C₁-C₄ alkyl radical, especially ethyl;
S represents a monosaccharide as described above;
X represents a group chosen from —CO—, —CH(OH)—, —C(NH₂)—, —C(CH₂CH₂CH₂OH)—, —C(NHPh)- and —C(CH₃)—, and more particularly a —CO—, —CH(OH)— or —CH(NH₂)— group, and preferentially a —CH(OH)— group.

Preferentially, a C-glycoside derivative of formula (I) is used, for which:
R denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X represents a group chosen from —CO—, —CH(OH)— and —CH(NH₂)— and preferentially a CH(OH)— group.

The salts that are acceptable for the non-therapeutic use of the compounds described in the present invention comprise conventional non-toxic salts of the said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates such as those formed during the final step of preparation of the said compounds due to the presence of solvents. Examples that may be mentioned include the solvates due to the presence of water or of linear or branched alcohols, for instance ethanol or isopropanol.

Among the C-glycoside derivatives of formula (I) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-α-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl 3-methyl-4-(acetamido-C-β-D-glucopyranosyl)butyrate;
152. ethyl 3-methyl-4-(acetamido-C-α-D-glucopyranosyl)butyrate;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As non-limiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may advantageously be used for the preparation of a composition according to the invention.

According to one particular embodiment, the C-glycoside derivative is C-β-D-xylopyranoside-2-hydroxypropane in the form of a solution containing 30% by weight of active material in a water/propylene glycol mixture (60%/40% by weight) such as the product manufactured by CHIMEX under the trade name Mexoryl SBB®.

Needless to say, according to the invention, a C-glycoside derivative corresponding to formula (I) may be used alone or as a mixture with other C-glycoside derivatives and in any proportion.

A C-glycoside derivative that is suitable for use in the invention may especially be obtained via the synthetic method described in document WO 02/051 828.

The amount of C-glycoside derivative to be used in a composition according to the invention depends on the desired cosmetic or therapeutic effect, and may thus vary within a wide range.

A person skilled in the art can readily determine the appropriate amounts, on the basis of his general knowledge.

A composition according to the invention may comprise a C-glycoside derivative in a proportion of from about 0.0001% to about 25% by weight of active material relative to the total weight of the composition, in particular from about 0.001% to about 10% by weight of active material and more particularly from about 0.05% to about 5% by weight of C-glycoside derivative active material relative to the total weight of the composition.

The mass ratio between the cosmetic or pharmaceutical compound liable to cause skin irritation and the C-glycoside derivative may range from 0.000004 to 70 000, or even from 0.001 to 5000 and preferably from 0.002 to 2000.

Additional Active Agents

The C-glycoside derivative used according to the invention may also be combined with at least 0.00001% to 95% by weight of an anti-inflammatory agent or another calmative, or a mixture thereof.

Examples of anti-inflammatory agents that may be mentioned include:
an inflammatory cytokine antagonist;
a steroidal anti-inflammatory agent (hydrocortisone, betamethasone, dexamethasone, etc.);
a non-steroidal anti-inflammatory agent, for instance aspirin or paracetamol; and mixtures thereof.

According to the invention, the term “inflammatory cytokine antagonists” means a compound capable of inhibiting the synthesis and/or the release of one or more inflammatory cytokines. Also included in the definition of an inflammatory cytokine antagonist are compounds that inhibit or block the binding of these cytokines to their receptor(s).

Examples that may be mentioned include antagonists of IL-1, of IL-8, of TNFα and of TNFβ, the tripeptide Lys-Pro-Val (KPV), and all the αMSH derivatives and related peptidometics characterized by activity of αMSH type by binding to the receptor or by control of the release of IL1, IL8 or TNFα, all the inhibitors of cytokine release (therapeutic class of CSAIDs for: cytokine suppressive anti-inflammatory drugs), the family of substituted pyrimidine N-oxides (EP99401719.2 (priority FR9809509) and EP 99402771.2 (priority FR9814211) lipoxin A4 inducers, algal extracts capable of modulating the production of cytokines by keratinocytes, for instance Phycosaccharide® sold by the company Codif, the water-glycol extract of the alga Laminaria saccharina, especially Phlorogine® sold by the company SECMA, extracts of Aloe vera or of Gingko biloba; the natural antagonist of IL-1 (IL-1RA) and the extract of the bark and roots of Terminalia sericea or Sericoside 3058500 sold by the company Indena.

According to one particular embodiment of the invention, the anti-inflammatory agent is chosen from algal extracts capable of modulating the production of cytokines by the keratinocytes, for instance Phycosaccharide® sold by the company Codif, the water-glycol extract of the alga Laminaria saccharina, especially Phlorogine® sold by the company SECMA, extracts of Aloe vera, the extract of the bark and roots of Terminalia sericea or Sericoside 3058500 sold by the company Indena.

The anti-inflammatory agents are preferably present in the compositions in accordance with the invention in a concentration that may range between 0.00001% and 10% by weight relative to the total weight of the composition. Even more preferentially, the concentration of anti-inflammatory compound may range between 0.0005% and 2% by weight relative to the total weight of the composition.

As examples of calmatives that may be used in the compositions of the invention, mention may be made of:
allantoin;
beta-glycyrrhetinic acid, extracts containing it, for instance the extract of Glycyrrhiza glabra (licorice), and complexes containing it, for instance the allantoin/glycyrrhetinic acid complex;
lyophilized or non-lyophilized planktons, extracts thereof and complexes thereof;
escin and plant extracts containing it, for instance the extract of common horse chestnut;
xanthine derivatives, for instance diethylaminoethyl theophylline hydrochloride;
waters and extracts (for example aqueous, aqueous-alcoholic or water-glycol extracts) of flowers and plants, for instance cornflower water, camomile water, mint water, lime water, rosewater, extracts of Rosacea plants (e.g.: Rosa gallica), extracts of peony, extracts of hawthorn, extracts of yarrow, extracts of mallow, extracts of marigold, extracts of sweet clover, extracts of sage, elderberry water, extracts of Ginkgo biloba, extracts of arnica, extracts of oregano, extracts of green tea, extracts of waterlily blossom, extracts of iris, extracts of birch bark and extracts of Aloe vera;
asiatic acid and plant extracts containing it, for instance Centella asiatica;
bisabolol;
fruit extracts, for instance extract of pineapple, extract of papaya; extract of guava;
algae, especially of the Laminaria type (for example red or brown algae) such as the extract of brown alga Padina pavonica, for instance HPS 3 Padina Pavonica sold by the company Alban Muller;
pyrrolidonecarboxylates and especially of zinc (Zn—PCA) or of copper (Cu—PCA);
oils of plant origin, for instance canola seed oil and shea butter oil;
essential oils, for example of coriander, of balm, of lavender, of mint or of camomile, and mixtures thereof;
acexamic acid and transexamic acid (4-trans-aminomethylcyclohexanecarboxylic acid);
ursolic acid and extracts containing it, for instance extract of rosemary leaf;
polysaccharides containing fucose, for instance Fucogel 1000 sold by Solabia (aqueous solution comprising 1% dry matter of polysaccharide comprising fucose, galactose and galacturonic acid);
electrolytes and in particular an aqueous mixture comprising from 30% to 35% of magnesium chloride, from 20% to 28% of potassium chloride, from 3% to 10% of sodium chloride, from 0.2% to 1% of calcium chloride, from 0.1% to 0.6% of magnesium bromide and from 0.1% to 0.5% of insoluble matter, the said mixture being referred to herein as Dead Sea bath salts, since it corresponds to the main salts contained in the Dead Sea;
galactolipids, for example derived from oat, for instance digalactosyl diglyceride or monogalactosyl diglyceride;
amino acids, derivatives thereof and salts thereof, such as the sodium salt of amino acids grafted onto cocoyl chains, sold in the form of a mixture under the name Sepicalm S by the company SEPPIC, capryloylglycine sold under the name Lipacide C8G by the company SEPPIC and the mixture of capryloylglycine, of cinnamon and of sarcosine sold under the name Sepicontrol A5 by the company SEPPIC;
TNFα antagonists such as lisophylline, A802715, sulfasalazine, CDP-571 (anti-TNFα antibody), and MDL-201112;
substance P antagonists such as sendide, spantide II and the peptides described in patent application EP-A-680 749, and extracts of filamentous bacteria described in patent application EP-A-761 204;
CGRP antagonists, such as CGRP 8-37, anti-CGRP antibodies, or plant extracts with CGRP-antagonist activity (e.g.: Iris pallida);
divalent strontium, zinc, manganese, magnesium or calcium salts, such as those described in documents WO-A-96/19184, WO-A-96/19182 and WO-A-96/19228; and mixtures thereof.

In particular, the other calmative may advantageously be chosen from allantoin, β-glycyrrhetinic acid, extracts containing it, for instance the extract of Glycyrrhiza glabra (licorice), and complexes containing it, for instance the allantoin/glycyrrhetinic acid complex; lyophilized or non-lyophilized planktons, extracts thereof and complexes thereof; waters and extracts of flowers and plants: camomile water, lime water, rosewater, birch extracts; bisabolol; essential oils, for example of coriander; algae, especially of the Laminaria type (for example red or brown algae) such as the extract of the brown alga Padina pavonica, for instance HPS 3 Padina Pavonica sold by the company Alban Muller; acexamic acid and transexamic acid (4-trans-aminomethylcyclohexanecarboxylic acid); ursolic acid and extracts containing it, for instance an extract of rosemary leaf; polysaccharides containing fucose, for instance Fucogel 1000 sold by the company Solabia; electrolytes and in particular an aqueous mixture such as the Dead Sea bath salts; amino acids, for instance Sepicalm S and VG from SEPPIC and divalent magnesium salts such as magnesium gluconate.

A subject of the invention is also a composition for topical application to the skin and/or mucous membranes and/or the scalp, comprising, in a physiologically acceptable medium:
0.00001% to 95% by weight of at least one anti-inflammatory and/or calmative compound, and
at least one C-glycoside derivative.

The anti-inflammatory and/or calmative compound is as described above.

According to a particular embodiment, the invention concerns a composition for topical application on the skin and/or a mucous membrane and/or scalp comprising, in a physiologically acceptable medium, at least a C-glycoside derivative and a rose extract, in particular of the genus Rosa.

According to this particular embodiment, le C-glycoside derivative is as defined above, inclusive in its more specific definitions and in particular is C-β-D-xylopyranoside-2-hydroxypropane, for example in the form of a solution containing 30% by weight of active material in a water/propylene glycol mixture (60%/40% by weight) such as the product manufactured by CHIMEX under the trade name Mexoryl SBB®.

The Rosa kind comprises more than 1000 species among which one can mention Rosa alba, Rosa alpina, Rosa canina, Rosa cinnamonea, Rosa gallica, Rosa repens, Rosa rubrifolia, Rosa rubiginosa, Rosa sempervirens, Rosa spinosissima, Rosa stylosa, Rosa tomentosa or Rosa villosa.

The extract of plant of the Rosa kind used according to the invention can be an extract prepared starting from material resulting from at least a plant pertaining to a species chosen among Rosa alba L, Rosa alpina, Rosa canina L, Rosa centifolia L, Rosa cinnamonea, Rosa damascena Mill., Rosa gallica L, Rosa repens, Rosa rubrifolia, Rosa rubiginosa, Rosa sempervirens, Rosa spinosissima, Rosa stylosa, Rosa tomentosa, Rosa villosa.

Preferably according to the invention, the plant belongs to the species Rosa gallica L.

The extract of at least a plant of the Rosa kind used according to the invention can be obtained starting from vegetable material resulting from whole plant or part of plant like the sheets, the stems, the flowers, the petals, the roots or of the undifferentiated cells.

According to the invention, the quantity in extract of the plant of the Rosa kind used in the composition is of course a function of the required effect and can thus vary on the whole.

Any method of known extraction of the person skilled in the art can be used to prepare the extract contained in the composition according to the invention.

One can in particular quote the aqueous, alcoholic extracts or using an organic solvent.

By aqueous solvent one understands any solvent constituted completely or partially of water. One can thus mention water itself, the alcoholic solvent in any proportion or solvents made up of water and a compound like the propylene glycol in any proportion. Among alcoholic solvents one can quote ethanol in particular. Whatever the mode of preparation used according to the invention, subsequent steps aiming at supporting the conservation and/or stabilization can be added without for that modifying the extract nature. Thus, for example the extract obtained can be freeze-dried by all traditional methods of freeze-drying. One can thus obtain a powder which can be used directly or mixed in a suitable solvent before use.

The vegetable extract of the Rosa kind as previously described used according to the invention can be present in the composition, in a content from 0.001 to 50% by weight, relative to the total weight of the composition, preferably from 0.01% to 30% by weight, and preferentially from 0.1 to 15% by weight.

According to a particular embodiment, the present invention relates to a composition comprising at least a C-glycoside derivative and an extract of Rosacea Rosa gallica.

Composition

The composition of the invention may be a cosmetic or dermatological composition.

The C-glycoside derivative according to the invention may be incorporated into the cosmetic or dermatological composition that contains a compound liable to cause skin irritation or, according to an alternative, may be incorporated into a separate calmative composition, which will be applied before and/or after the composition containing the compound liable to cause skin irritation depending on whether it is desired to initiate the inflammatory phase, which may be useful for certain applications (vasodilation, hair removal, etc.), or, on the contrary, prevent it.

In the case of certain desquamating agents or peeling agents, for example, it may in fact be advantageous to apply, in a first stage, the composition containing the said peeling agent intended to induce cutaneous acellular pro-inflammatory changes (vasodilation) to remodel or remove hair from the epidermis, and then, in a second stage, to apply a calmative composition containing a C-glycoside derivative, the said C-glycoside derivative being intended to prevent the acellular pro-inflammatory events from being relayed by a step of cellular mobilization and chemoattraction, which may lead to an acute inflammatory reaction and a phase of undesirable chronicity involving immunological mechanisms that may be deleterious when they turn against the body.

The physiologically acceptable medium may comprise water, organic solvents such as a C₁-C₈ alcohol, especially ethanol, isopropanol, tert-butanol or n-butanol; a polyol such as glycerol; a glycol, for instance butylene glycol, isoprene glycol, propylene glycol or polyethylene glycols such as PEG-8; polyol ethers, acetone and ethyl acetate.

The composition of the invention may thus constitute a composition for treating or caring for the skin (including the scalp), keratin fibres (hair, eyelashes or eyebrows), the nails or the lips, or an antisun or artificial tanning composition, or a product for cleansing or for removing makeup from the skin, the hair, the eyebrows or the eyelashes, a deodorant product or a fragrancing compound. It is then generally uncoloured or weakly coloured, and it may optionally contain cosmetic or dermatological active agents. It may then be used as a care base for the skin or the lips (lip balms, for protecting the lips against the cold and/or sunlight and/or the wind), as a day or night care cream for facial and/or bodily skin. It may also be in the form of a colouring or non-colouring, medicated or unmedicated shampoo, and of a hair-conditioning compound.

The composition according to the invention may also constitute a coloured cosmetic composition and especially a makeup composition for the skin, keratin fibres (hair or eyelashes) and/or mucous membranes, in particular a foundation, a blusher, a makeup rouge, an eye shadow, a mascara, an eyeliner, a concealer compound in stick form, a nail varnish, a lipstick or a lip gloss, optionally having care or treatment properties, or a body tattoo.

Thus, a subject of the invention is also the cosmetic use of the composition as defined above, for caring for, treating, cleansing and/or making up keratin materials (skin, including the scalp, keratin fibres and mucous membranes).

The composition according to the invention may be in the galenical forms conventionally used for topical application and especially in the form of dispersions of the lotion or serum type, emulsions of liquid or semi-liquid consistency of the milk type, obtained by dispersing a fatty phase in an aqueous phase (O/W) or conversely (W/O), or suspensions or emulsions of soft, semi-solid or solid consistency of the cream or gel type, or alternatively multiple emulsions (W/O/W or O/W/O), microemulsions, nanoemulsions, vesicular dispersions of ionic and/or nonionic type, or wax/aqueous phase dispersions. These compositions are prepared according to the usual methods.

It may also be in the form of a transdermal system allowing active or passive transdermal release of the active agent(s), for example of patch type or of patch gel type (hydrogel).

When the composition is in emulsion form, the proportion of the oily phase of the emulsion may range, for example, from 5% to 80% by weight and preferably from 5% to 50% by weight relative to the total weight of the composition. The oils, emulsifiers and coemulsifiers used in the composition in emulsion form are chosen from those conventionally used in cosmetics or dermatology.

The emulsifier and the coemulsifier are generally present in the composition in a proportion ranging from 0.3% to 30% by weight and preferably from 0.5% to 20% by weight relative to the total weight of the composition. The emulsion may also contain lipid vesicles.

Moisturizers may also be used, among which mention may be made especially of:
glycerol,
hydroxyethylurea or Hydrovance® from National Starch,
polyols,
amino acids and N-acyl derivatives thereof,
pyrrolidonecarboxylic acid and salts and N-acyl derivatives thereof,
sugars, alkyl ethers thereof and alkyl esters thereof,
heterogeneous polyholosides,
trehalose,
ectoin,
glycosaminoglycans and sulfates thereof,
phospholipids and polymers containing phosphorylcholine groups,
cholesterol,
phytosterols, and
hyaluronic acid.

Examples of oils that will be used include hydrocarbon-based oils of plant origin, synthetic esters and ethers, and silicone oils, and mixtures thereof.

Surfactants may also be added to homogenize the mixture of the aqueous and oily phases.

The examples that follow are given as non-limiting illustrations of the field of the invention.

The C-glycoside derivative used is C-β-D-xylopyranoside-2-hydroxypropane sold by the company Chimex under the trade name Mexoryl SBB®. It is in the form of a solution containing 30% by weight of active material in a 60/40 water/propylene glycol mixture.

### EXAMPLES

### Example 1

### Evaluation of the Anti-Irritant Effect of a C-Glycoside Compound

The test consists in evaluating the effect of C-β-D-xylopyranoside-2-hydroxypropane on inhibition of the response of the surface epidermal cells to an agent liable to cause skin irritation, retinol, by assaying the interleukin-8 secreted by normal human epidermal keratinocytes (NHEK) after stimulation with the agent liable to cause skin irritation.

Principle and aim of the study:

This study uses the test developed by Wilmer [Wilmer, J. L. et al., J. invest. Dermatol., 102: 915-922 (1994)]. This test allows evaluation of the anti-irritant potential of various molecules, on a human keratinocyte (NHEK) cell line. In this test, an irritant situation is mimicked by exacerbating the production of IL-8 by the NHEKs by addition of retinol to the culture medium, Il-8 being involved in the initiation of keratinocyte irritation. Next, the anti-irritant effect of a C-glycoside compound is measured by means of its inhibitory action with respect to this exacerbated production.

Experimental conditions:

Normal human epidermal keratinocytes are incubated in the presence of 10⁻⁶ M retinol for 24 hours at 37° C. In response to the retinol, the production of the chemotactic agent interleukine 8 (IL-8) is measured via enzymatic assay using an assay kit (Elisa D8050) sold by the company R&D. The absorbance values are measured with a microplate reader (MRX/Dynatech) according to the procedures supplied with the assay kit. To evaluate the anti-irritant protective effect of the said C-glycoside compound, the production of the chemotactic marker IL-8 by the human keratinocytes is measured in the presence of various concentrations greater than or equal to 0.04% of C-glycoside active material.

A control test is also performed with dexamethasone, which is a reference calmative.

The results are expressed as a percentage of the control values after subtracting the background noise and as a percentage of inhibition of these values obtained in the presence of the compounds.

Assay of IL8 without stimulation

| | Concen- | | | | |
|---|---|---|---|---|---|
| Treatment | tration | IL8 (pg/ml) | % blank | p | % viability |
| Blank | — | 272.1 | 100 | — | 100 |
| C-β-D-xylopyranoside- | 2% | 145.2 | 53 | P < 0.01 | 97 |
| 2-hydroxypropane | | | | | |

Assay of IL8 + stimulation with retinol

| | Concen- | | % blank | | |
|---|---|---|---|---|---|
| Treatment | tration. | IL8 (pg/ml) | (+retinol) | P | % viability |
| Blank (+retinol) | — | 206.6 | 100 | — | 98 |
| Retinol-free control | | 129.6 | 63 | P < 0.01 | 100 |
| Dexamethasone (+retinol) | 10⁻⁷M | 117.7 | 57 | P < 0.01 | 99 |
| C-β-D-xylopyranoside- | 2% | 124.4 | 60 | P < 0.01 | 104 |
| 2-hydroxypropane (+retinol) | | | | | |

These results clearly show that C-β-D-xylopyranoside-2-hydroxypropane reduces the irritant reaction of retinol.

### Example 2

### Serum

| Chemical name | weight % |
|---|---|
| Lactic acid | 10 |
| Glycerol | 3 |
| C-β-D-xylopyranoside-2-hydroxypropane as | 3 (AM) |
| a solution at 30% by weight in a 60/40 | |
| water/propylene glycol mixture | |
| 96° ethyl alcohol, not denatured | 10 |
| Sterilized demineralized water | qs 100 |

### Example 3

### O/W Emulsion

| Phase | Chemical name | weight % |
|---|---|---|
| A | Preserving agent | qs |
| | Disodium salt of ethylenediaminetetraacetic acid | 0.15 |
| | dihydrate | |
| | Glycerol | 3 |
| | C-β-D-xylopyranoside-2-hydroxypropane as a | 3 (AM) |
| | solution at 30% by weight in a 60/40 water/propylene | |
| | glycol mixture | |
| | Sterilized demineralized water | 55.22 |
| B | Mixture of glyceryl monostearate and polyethylene | 0.3 |
| | glycol (100 EO) stearate (50/50): Arlacel 165 | |
| | Pure cetyl alcohol, of natural origin | 0.4 |
| | Preserving agent | qs |
| C | Retinol | 1 |
| | Isopropyl N-lauroylsarcosinate: ELDEW SL-205 | 10 |
| | from Ajinomoto | |
| D | Acrylic acid/stearyl methacrylate copolymer | 0.5 |
| | polymerized in an ethyl acetate/cyclohexane mixture: | |
| | CARBOPOL 1382 from NOVEON | |
| | Cyclohexadimethylsiloxane (viscosity: 8 cSt) | 5 |
| E | Sterilized demineralized water | 10 |
| | 96° ethyl alcohol, not denatured | 5 |
| F | Acrylamide/sodium acrylamido- | 1 |
| | 2-methylpropanesulfonate copolymer as an inverse | |
| | emulsion at 40% in isoparaffin/water: Sepigel 305 | |
| | from SEPPIC | |
| H | 99% triethanolamine | 1.03 |
| | Sterilized demineralized water | 7 |

### Example 4

### After-Sun Calmative Gel

| Phase | Chemical name | weight % |
|---|---|---|
| A | Glycerol | 7 |
| | Allantoin | 1 |
| | Preserving agent | qs |
| | Sequestrant | qs |
| | C-β-D-xylopyranoside-2-hydroxypropane as a | 3 (AM) |
| | solution at 30% by weight in a 60/40 | |
| | water/propylene glycol mixture | |
| | Water | qs 100 |
| B | Petroleum jelly | 10 |
| | Mixture of cocoyl polyglucoside and of cetyl stearyl | 3 |
| | alcohol: Montanov 82 from SEPPIC | |
| | Preserving agent | qs |
| | Plant oil | 3 |
| | Cyclopentasiloxane | 5 |
| | Vitamin E | 0.1 |
| C | Xanthan gum | 0.2 |
| | Partially neutralized polyacrylamidomethylpropane- | 0.5 |
| | sulfonic acid: Hostacerin AMPS from Clariant | |

### Example 5

### Transparent Exfoliant Gel

| Phase | Chemical name | weight % |
|---|---|---|
| A | Sodium acrylamido-2-methylpropanesulfonate/ | 2 |
| | hydroxyethyl acrylate copolymer: SEPINOV EMT 10 | |
| | Glycerol | 4 |
| | Propylene glycol | 4 |
| | Sequestrant | qs |
| | Preserving agent | qs |
| | Dye | qs |
| | Alcohol | 10 |
| | C-β-D-xylopyranoside-2-hydroxypropane as a solution | 1.5 (AM) |
| | at 30% by weight in a 60/40 water/propylene glycol | |
| | mixture | |
| | Water | qs 100 |
| B | Glycolic acid | 2 |
| | Water | 10 |
| C | Fragrance | qs |

### Example 6

### Cream Deodorant

| Phase | Chemical name | weight % |
|---|---|---|
| A | Cetylstearyl alcohol (30/70 C16/C18) | 3 |
| | Oxyethylenated (33 EO) cetylstearyl alcohol | 0.5 |
| | Fragrance | qs |
| | Cyclohexasiloxane | 1 |
| B | Preserving agent | qs |
| | Sequestrant | qs |
| | Aluminium hydrochloride | 15 |
| | C-β-D-xylopyranoside-2-hydroxypropane as a | 2.4 (AM) |
| | solution at 30% by weight in a 60/40 water/propylene | |
| | glycol mixture | |
| | Water | qs 100 |

### Example 7

### Facial Cleanser

| Chemical name | weight % |
|---|---|
| Aqueous 70% sodium lauryl ether sulfate solution | 8 |
| Aqueous 30% cocoylbetaine solution | 8 |
| Lauryl ether carboxylic acid (10 EO) | 1 |
| Glycerol | 4 |
| Hexylene glycol | |
| Plant oil | 0.1 |
| Vitamin E | 0.3 |
| Sequestrant | qs |
| Preserving agent | qs |
| C-β-D-xylopyranoside-2-hydroxypropane as a solution at 30% | 1.5 (AM) |
| by weight in a 60/40 water/propylene glycol mixture | |
| Fragrance | qs |
| Water | qs 100 |

AM: Active Material.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Non-therapeutic method to prevent and/or treat skin in need of a calmative, comprising at least a step of applying to the skin a composition comprising at least one C-glycoside derivative.

2. Method according to claim 1, for preventing and/or treating skin reactions chosen from redness, itching, heating sensations, burning sensations, stinging and tautness.

3. Method according to claim 1, wherein the said C-glycoside derivative is intended for preventing and/or reducing the said skin reaction induced by at least one condition chosen from the action of xenobiotics, chemical products, compounds liable to cause skin irritation, a peeling treatment, the action of temperature, the climate, UV radiation or atmospheric pollution, or by friction.

4. Method according to claim 3, wherein the said C-glycoside derivative is intended for preventing and/or reducing the irritant effect of a cosmetic or dermatological composition containing one or more compounds liable to cause skin irritation.

5. Method according to claim 4, wherein the said C-glycoside derivative is incorporated into a separate composition intended to be applied after a composition containing the compound(s) liable to cause skin irritation.

6. Method according to claim 4, wherein the said C-glycoside derivative is incorporated into a composition containing the compound(s) liable to cause skin irritation.

7. Method according to claim 4, wherein the compound liable to cause skin irritation is chosen from cosmetic active agents, dermatological active agents, surfactants, fragrances, solvents, propellants, preserving agents and detergents, and mixtures thereof.

8. Method according to claim 7, wherein the compound liable to cause skin irritation is a cosmetic or dermatological active agent chosen from the group of desquamating agents, retinoids, vitamin D and derivatives thereof, vitamin B9 and derivatives thereof, urea and derivatives thereof, peroxides, hair-loss counteractants, hair dyes and hair colorants, antiperspirants, hair-removing and/or permanent-waving active agents, thioglycolate and salts thereof, phenoxyethanol, 1,2-pentanediol, fragrancing alcoholic solutions, anthralins, anthranoids, lithium salts, depigmenting agents, slimming active agents with a heating effect, nicotinates and derivatives thereof, capsaicin, anti-louse active agents, antiproliferative agents, antiviral agents, antiparasitic agents, antifungal agents, anti-pruriginous agents, anti-seborrhoeic agents, sunscreens and pro-pigmenting agents, and mixtures thereof, or is an anionic, cationic or amphoteric surfactant.

9. Method according to claim 7, wherein the compound liable to cause skin irritation is chosen from retinoids, α-hydroxy acids, β-hydroxy acids, saturated and unsaturated dicarboxylic acids such as octadecenedioic acid, anionic, cationic or amphoteric surfactants, 5-n-octanoylsalicylic acid, antiperspirant active agents such as aluminium salts, (N-2-hydroxyethylpiperazine-N-2-ethane)sulfonic acid and cinnamic acid, and mixtures thereof.

10. Method according to claim 4, wherein the compound(s) liable to cause skin irritation is present in a content ranging from 0.0001% to 70% by weight relative to the total weight of the composition.

11. A non therapeutic method for the treatment of sensitive skin and/or scalp comprising at least a step of applying to the skin and/or scalp a composition comprising at least one C-glycoside derivative.

12. A non therapeutic method for the treatment of sensitive skin and/or scalp, comprising at least a step of applying to the skin and/or scalp a composition comprising at least one C-glycoside derivative, wherein it is intended for the treatment of redness and/or darters and/or dysaesthetic sensations chosen among stinging sensations, pins and needles, itching, burning, overheating, discomfort, tautness of of sensitive skin and/or scalp.

13. A non therapeutic method for the treatment of sensitive skin and/or scalp comprising at least a step of applying to the skin and/or scalp a composition comprising at least one C-glycoside derivative, wherein the C-glycoside derivative is associated with a rose extract.

14. A dermatological method for preventing and/or treating skin disorders associated with skin disorders associated with skin irritation comprising at least a step of applying to the skin a composition comprising at least one C-glycoside derivative.

15. A dermatological method according to claim 14, wherein skin disorders associated with skin irritation are encountered in the case of individuals with irritable and/or allergic skin and/or mucous membranes and/or scalp.

16. Method according to claim 14, wherein the said C-glycoside derivative is intended for preventing and/or reducing the irritant effect of a cosmetic or dermatological composition containing one or more compounds liable to cause skin irritation.

17. Method according to claim 14, wherein the said composition is intended for treating skin disorders associated with a skin irritation of inflammatory or immunoallergic type.

18. Method according to claim 14, wherein the skin disorders are chosen from dry patches, oedema and/or spots, inflammatory erythema; pruritus; psoriasis, cutaneous atopy, atopic dermatitis; urticaria; contact dermatitis, eczema; seborrhoeic dermatitis; acne; inflammatory hyperpigmentations; immune dermatitis; bullous immune diseases; scleroderma; actinic elastosis; pelada or alopecia areata; vitiligo; systemic lupus erythematosus; pemphigus vulgaris; dystrophic bulbous epidermolysis and baldness of autoimmune origin.

19. Method according to claim 1, wherein the C-glycoside derivative corresponds to the general formula (I) below: 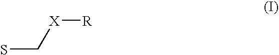
in which:
R represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀, or saturated or unsaturated, branched or cyclic C₃-C₂₀ linear hydrofluoroalkyl or perfluoroalkyl radical; the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ or unsaturated C₂-C₃₀, or a saturated or unsaturated, branched or cyclic C₃-C₃₀ linear alkyl, perfluoroalkyl or hydrofluoroalkyl radical; or a C₆-C₁₀ aryl radical,
X represents a radical chosen from the groups: 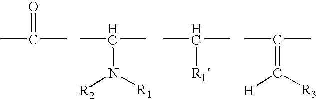
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be α or β, and also the cosmetically acceptable salts thereof, the solvates thereof such as hydrates, and the isomers thereof.

20. Method according to claim 19, wherein S represents a monosaccharide chosen from D-glucose, D-xylose, L-fucose, D-galactose and D-maltose, especially D-xylose.

21. Method according to claim 19, wherein X represents a group chosen from —CO—, —CH(OH)— and —CH(NH₂)— group.

22. Method according to claim 19, wherein R denotes a linear C₁-C₄, optionally substituted with —OH, —COOH or —COOR″₂, R″₂ being a saturated C₁-C₄ alkyl radical.

23. Method according to claim 1, wherein the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

24. Method according to claim 1, wherein the C-glycoside derivative is chosen from C-β-D-xylopyranoside-2-hydroxypropane and C-α-D-xylopyranoside-2-hydroxypropane, and is more particularly C-β-D-xylopyranoside-2-hydroxypropane.

25. Method according to claim 1, wherein the C-glycoside derivative is present in a content ranging from about 0.0001% to about 25% by weight of active material relative to the total weight of the composition.

26. Method according to claim 1, wherein the C-glycoside derivative is combined with at least one anti-inflammatory agent or a calmative, or a mixture thereof.

27. Method according to claim 1, wherein the composition is intended for treating irritable and/or allergic skin and/or mucous membranes and/or scalps.

28. Method according to claim 14, wherein the C-glycoside derivative corresponds to the general formula (I) below: 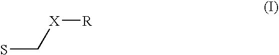
in which:
R represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀, or saturated or unsaturated, branched or cyclic C₃-C₂₀ linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ or unsaturated C₂-C₃₀, or a saturated or unsaturated, branched or cyclic C₃-C₃₀ linear alky, perfluoroalkyl or hydrofluoroalkyl radical; or a C₆-C₁₀ aryl radical,
X represents a radical chosen from the groups: 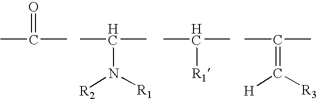
with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide chosen from D-glucose, D-xylose, L-fucose, D-galactose and D-maltose, especially D-xylose, and
the bond S—CH₂—X represents a bond of C-anomeric nature, which may be α or β and also the cosmetically acceptable salts thereof, the solvates thereof such as hydrates, and the isomers thereof.

29. Composition for topical application to the skin and/or mucous membranes and/or the scalp, comprising, in a physiologically acceptable medium, at least one C-glycoside derivative and at least one cosmetic or pharmaceutical compound liable to cause skin irritation, chosen from desquamating agents, retinoids, urea and derivatives thereof, vitamin D and derivatives thereof, vitamin B9 and derivatives thereof, peroxides, hair-loss counteractants, hair dyes and hair colorants, antiperspirants, deodorants, hair-removing and/or permanent-waving active agents, thioglycolate and salts thereof, phenoxyethanol, 1,2-pentanediol, anthralins, anthranoids, lithium salts, depigmenting agents, certain slimming active agents with a heating effect, nicotinates and derivatives thereof, capsaicin, anti-louse active agents, antiproliferative agents, antiviral agents, antiparasitic agents, antifungal agents, anti-pruriginous agents, anti-seborrhoeic agents, certain sunscreens, pro-pigmenting agents, alkyl sulfates and alkyl ether sulfates, and cationic or amphoteric surfactants.

30. Composition according to claim 29, wherein the compound liable to cause skin irritation is chosen from retinoids, α-hydroxy acids, β-hydroxy acids, saturated and unsaturated dicarboxylic acids such as octadecenedioic acid, cationic or amphoteric surfactants, 5-n-octanoylsalicylic acid, antiperspirant active agents such as aluminium salts, (N-2-hydroxyethylpiperazine-N-2-ethane)sulfonic acid and cinnamic acid, and mixtures thereof.

31. Composition according to claim 29, wherein the C-glycoside derivative is present in a content ranging from about 0.0001% to about 25% by weight of active material relative to the total weight of the composition.

32. Composition according to claim 29, wherein the compound(s) liable to cause skin irritation is (are) present in a content ranging from 0.0001% to 70% by weight relative to the total weight of the composition.

33. Composition according to claim 29, wherein it also comprises at least one anti-inflammatory agent or a calmative, or a mixture thereof.

34. Composition for topical application to the skin and/or mucous membranes and/or the scalp, comprising, in a physiologically acceptable medium:
0.00001% to 95% by weight of at least one anti-inflammatory and/or calmative compound, and
at least one C-glycoside derivative.

35. Composition according to claim 34, wherein the anti-inflammatory compound is chosen from algal extracts capable of modulating the production of cytokines by the keratinocytes, extracts of Aloe vera, and an extract of the bark and roots of Terminalia sericea, and the calmative compound is chosen from allantoin, β-glycyrrhetinic acid, extracts containing it, for instance an extract of Glycyrrhiza glabra, and complexes containing it, for instance the allantoin/glycyrrhetinic acid complex; lyophilized or non-lyophilized planktons, extracts thereof and complexes thereof; waters and extracts of flowers and plants: camomile water, lime water, rosewater, birch extracts; bisabolol; essential oils, for example of coriander; algae, especially of the Laminaria type, such as an extract of the brown alga Padina pavonica, acexamic acid and transexamic acid; ursolic acid and extracts containing it, for instance an extract of rosemary leaf; polysaccharides containing fucose; electrolytes; amino acids, and divalent magnesium salts such as magnesium gluconate.

36. Composition according to claim 35, wherein it comprises an extract of Rosacea Rosa gallica.

37. Cosmetic process for the non-therapeutic care of the skin, the hair and/or the scalp, wherein it comprises at least one step that consists in applying to the skin, the hair and/or the scalp a cosmetic composition as defined according to claim 34.

38. Cosmetic process according to claim 37, wherein the composition is applied to an irritable and/or allergic skin, mucous membrane and/or scalp.

39. Cosmetic process according to claim 37, wherein it is intended for preventing and/or treating the signs of ageing of the skin, and/or for lightening the skin, and/or for reviving the radiance of the complexion, and/or for stimulating hair growth or preventing hair loss.
